SEQUENCE LISTING (4 sequences)

SEQ ID NO: 1 - DNA, 1100 bp, Artificial Sequence
  nucleotide sequence of the circular RNA-0007535
tttcagaaagtgctttctctctgtggacatgaggattggattagaggagtggaatgggcagcctttggtagagatcttttcctagcaagctgttcacaagattgcctgataagaatatggaagctgtatataaagtcaacatctttagaaactcaggatgacgataacataagactgaaagaaaatacttttaccatagaaaatgaaagtgttaaaatagcatttgctgttactctggagacagtgctagccggtcatgaaaactgggtaaatgcagttcactggcaacctgtgttttacaaagatggtgtcctacagcagccagtgagattattatctgcttccatggataaaaccatgattctctgggctccagatgaagagtcaggagtttggctagaacaggttcgagtaggtgaagtaggtgggaatactttgggattttatgattgccagttcaatgaagatggctccatgatcattgctcatgctttccacggagcgttgcacctttggaaacagaatacagttaacccaagagagtggactccagagattgtcatttcaggacactttgatggtgtccaagacctagtctgggatccagaaggagaatttattatcactgttggtactgatcagacaactagactttttgctccatggaagagaaaagaccaatcacaggtgacttggcatgaaattgcaaggcctcagatacatgggtatgacctgaaatgtttggcaatgattaatcggtttcagtttgtatctggagcagatgaaaaagttcttcgggttttttctgcacctcggaattttgtggaaaatttttgtgccattacaggacaatcactgaatcatgtgctctgtaatcaagatagtgatcttccagaaggagccactgtccctgcattgggattatcaaataaagctgtctttcagggagatatagcttctcagccttctgatgaagaggagctgttaactagtactggttttgagtatcagcaggtggcctttcagccctccatacttactgagcctcccactgaggatcatcttctgcagaatactttgtggcctgaagttcaaaaact

SEQ ID NO: 2 - DNA, 19 bp, Artificial Sequence
  target gene for synthetic interfering double-stranded RNA
gaagttcaaaaacttttca

SEQ ID NO: 3 - DNA, 23 bp, Artificial Sequence
  forward primer
cctttcagccctccatacttact

SEQ ID NO: 4 - DNA, 25 bp, Artificial Sequence
  reverse primer
ccatattcttatcaggcaatcttgt

(19) United States
(12) Patent Application Publication
(10) Pub. No.: US 2022/0339182 A1
(43) Pub. Date: Oct. 27, 2022

(54) USE OF REAGENT FOR DOWN-REGULATING CIRCULAR GENE EXPRESSION IN PREPARATION OF DRUGS FOR PREVENTING AND/OR TREATING PULMONARY FIBROSIS AND DRUG THEREOF

(71) Applicant: BINZHOU MEDICAL UNIVERSITY, Yantai City, Shandong (CN)

(72) Inventors: Xiaodong SONG, Yantai City, Shandong (CN); Ming'e LI, Yantai City, Shandong (CN); Jinjin ZHANG, Yantai City, Shandong (CN); Pan XU, Yantai City, Shandong (CN); Hongbo LI, Yantai City, Shandong (CN); Rongrong LI, Yantai City, Shandong (US)

(21) Appl. No.: 17/642,218

(22) PCT Filed: Jan. 4, 2021
(86) PCT No.: PCT/CN2021/000001

§ 371 Date: Mar. 10, 2022

(30) Foreign Application Priority Data
Jan. 2, 2020 (CN) 202010001352.4

(51) Int. Cl. A61K 31/713; A61P 11/00; C12N 15/113

(52) U.S. Cl. CPC A61K 31/713; A61P 11/00; C12N 15/113; C12N 2310/14

(57) ABSTRACT

Disclosed is use of a reagent for down-regulating the circular RNA-0007535 expression in preparation of drugs for preventing and/or treating pulmonary fibrosis and drugs for down-regulating the circular RNA-0007535 expression, belonging to the technical field of pharmaceutical preparation. The present disclosure provides a use of a reagent for down-regulating the circular RNA-0007535 expression in preparation of drugs for preventing and/or treating pulmonary fibrosis, the nucleotide sequence of the circular RNA-0007535 is set forth in SEQ ID NO. 1. Drugs prepared from the reagent for down-regulating the circular RNA-0007535 expression may prevent and/or treat pulmonary fibrosis, the cyclic RNA-0007535, as a potential molecular and drug target for treating pulmonary fibrosis diseases, provides a new perspective and field for exploring a regulation mechanism of gene expression in the occurrence and development of pulmonary fibrosis and searching for an intervention/drug, and has good application prospects.

### CROSS REFERENCE TO RELATED APPLICATION

This patent application claims the benefit and priority of Chinese Patent Application NO. 202010001352.4 entitled “Use of reagent for down-regulating circular gene expression in preparation of drugs for preventing and/or treating pulmonary fibrosis and drug thereof” filed on Jan. 2, 2020, the entire contents of which are incorporate herein by reference.

### TECHNICAL FIELD

The present disclosure relates to the technical field of pharmaceutical preparation, and particularly relates to use of a reagent for down-regulating the circular RNA-0007535 expression in preparation of drugs for preventing and/or treating pulmonary fibrosis and drugs for down-regulating the circular RNA-0007535 expression.

### BACKGROUND ART

Idiopathic pulmonary fibrosis (IPF) is an interstitial lung disease in which lung injury occurs in the absence of a clear etiology, and progresses gradually, eventually leading to respiratory failure and death. The pathogenesis of IPF is still unclear, and may be related to the fibrous proliferation caused by the alveolar epithelial cells injury and lung abnormal repair due to the interaction of genetic factors and environmental exposure. IPF is characterized by cough, irreversible dyspnea, decreased lung function and respiratory failure, etc. The imaging examination shows bilateral reticular opacities of bronchiectasis and subpleural honeycomb changes Patients with atypical imaging examinations require lung biopsy to determine the nature of their lesions. There is no drug with definite efficacy for this disease. Lung transplantation is the only treatment for end-stage pulmonary fibrosis, but its application is limited by the high cost and difficult donor source. The use of genomics technology to study the molecular mechanisms and new targets of pulmonary fibrosis has become a research hotspot.

At present, the research on the participation of non-coding RNA in the regulation of pulmonary fibrosis diseases mainly focuses on the fields of miRNA and lncRNA, while the research on circRNA, such as ciR-012091 and circZC3H4 RNA, is rare, and there is almost no the research on circRNA in idiopathic pulmonary fibrosis.

### SUMMARY OF THE INVENTION

The purpose of the present disclosure is to provide a use of a reagent for down-regulating the circular RNA-0007535 expression in preparation of drugs for preventing and/or treating pulmonary fibrosis and drugs for down-regulating the circular RNA-0007535 expression. Drugs for down-regulating the circular RNA-0007535 expression are able to prevent and/or treat pulmonary fibrosis.

The present disclosure provides a use of a reagent for down-regulating the circular RNA-0007535 expression in preparation of drugs for preventing and/or treating pulmonary fibrosis, the nucleotide sequence of the circular RNA-0007535 is set forth in SEQ ID NO. 1.

The present disclosure also provides a use of a reagent for down-regulating the circular RNA-0007535 expression in preparation of drugs for preventing and/or treating pulmonary fibrosis by regulating a Hippo signaling pathway by adsorbing miR-630, and the nucleotide sequence of the circular RNA-0007535 is set forth in SEQ ID NO. 1.

The present disclosure also provides a use of a reagent for down-regulating the circular RNA-0007535 expression in preparation of drugs for inhibiting the differentiation of lung fibroblasts, and the nucleotide sequence of the circular RNA-0007535 is set forth in SEQ ID NO. 1.

The present disclosure also provides a use of a reagent for down-regulating the circular RNA-0007535 expression in preparation of drugs for inhibiting the activation of lung fibroblasts, the nucleotide sequence of the circular RNA-0007535 is set forth in SEQ ID NO. 1.

The present disclosure also provides a use of a reagent for down-regulating the circular RNA-0007535 expression in preparation of drugs for inhibiting the proliferation of lung fibroblasts, and the nucleotide sequence of the circular RNA-0007535 is set forth in SEQ ID NO. 1.

The present disclosure also provides a use of a reagent for down-regulating the circular RNA-0007535 expression in preparation of drugs for inhibiting the migration of lung fibroblasts, and the nucleotide sequence of the circular RNA-0007535 is set forth in SEQ ID NO. 1.

The present disclosure also provides a drug for down-regulating the circular RNA-0007535 expression, wherein the nucleotide sequence of the circular RNA-0007535 is set forth in SEQ ID NO. 1, and the drug includes interfering double-stranded RNA, the interfering double-stranded RNA is synthesized according to the nucleotide sequence set forth in SEQ ID NO. 2, and the interfering double-stranded RNA has the following structure:

```
          5′ GAAGUUCAAAAACUUUUCA dTdT 3′
                |||||||||||||||||||
3′ dTdT CUUCAAGUUUUUGAAAAGU 5′;
```

wherein each “|” represents a base pair.

The present disclosure provides a use of a reagent for down-regulating the circular RNA-0007535 expression in preparation of drugs for preventing and/or treating pulmonary fibrosis. According to the present disclosure, it is found that the circular RNA-0007535 can regulate the Hippo signaling pathway by adsorbing the miR-630, is a potential molecular and drug target for treating pulmonary fibrosis diseases, the present disclosure provides a new perspective and field for exploring a regulation mechanism of gene expression in the occurrence and development of pulmonary fibrosis and searching for an intervention/drug, and the circular RNA-0007535 has good application prospects. The experimental results shows that the circular RNA-0007535 (circRNA-0007535) is closely related to the occurrence of pulmonary fibrosis. Reduction of the circRNA-0007535 expression can inhibit the transdifferentiation of MRC-5 cells. The experimental results also shows that the proliferation and migration abilities of cells in the MRC-5/siRNA-0007535 group were significantly weakened. Reduction of the circRNA-0007535 expression can inhibit the activation, proliferation and migration of fibroblasts, and the circRNA-0007535 can be used as a molecular and drug target for the treatment of pulmonary fibrosis.

### BRIEF DESCRIPTION OF THE DRAWINGS

FIG. 1A shows the in vitro model expression level of circRNA-0007535 provided by the disclosure. FIG. 1B shows the in vivo expression level of circRNA-0007535 provided by the disclosure.

FIG. 2A-FIG. 2E show the effect of circRNA-0007535 provided by the present disclosure on the activation and function of myofibroblasts. FIG. 2A shows the expression level of circRNA-0007535 after different si-circRNA-0007535 transfection, FIG. 2B shows the transfection efficiency of the selected si-circRNA-0007535, FIG. 2C shows the transfection efficiency of the circRNA-0007535 over-expression vector, FIG. 2D shows the changes of pulmonary fibrosis related protein index after the over-expression of circRNA-0007535 detected by western blot, and FIG. 2E shows the changes of pulmonary fibrosis related protein index after the transfection of the interfering fragment si-circRNA-0007535 detected by western blot.

FIG. 3A-FIG. 3D show the ability of circRNA-0007535 provided by the present disclosure to regulate the proliferation and migration of myofibroblasts. FIG. 3A shows the proliferation ability of myofibroblasts after the over-expression of circRNA-0007535 detected by RTCA system, and FIG. 3B shows the proliferation ability of myofibroblasts after the transfection of the interference fragment si-circRNA-0007535 detected by RTCA system, FIG. 3C shows the migration ability of myofibroblasts after over-expression of circRNA-0007535 detected by the RTCA system, and FIG. 3D shows the migration ability of myofibroblasts after transfection of the interfering fragment si-circRNA-0007535 detected by the RTCA system.

FIG. 4A-FIG. 4B show the targeting relationship between miR-630 and circRNA-0007535 provided by the present disclosure. FIG. 4A shows the interaction between circRNA-0007535 and its target miRNA in an enlarged network, and FIG. 4B shows that the dual-luciferase report gene detection proved the miR-630 directly binds to circRNA-0007535.

FIG. 5A-FIG. 5D show the effect of circRNA-0007535 provided by the present disclosure on the activation and function of myofibroblasts. FIG. 5A shows the expression level of miR-630 in MRC-5 cells induced by TGF-β1 was decreased from 0 h to 72 h evaluated by qRT-PCR, and FIG. 5B shows the expression level of circRNA-0007535 after the addition of miR-630 mimic/inhibitor evaluate by qRT-PCR, FIG. 5C shows the changes of pulmonary fibrosis related protein indicators after transfection of miR-630 mimic detected by Western blot, and FIG. 5D shows the changes of pulmonary fibrosis related protein indicators after transfection of miR-630 inhibitor detected by Western blot.

FIG. 6A-FIG. 6D show the ability of miR-630 provided by the present disclosure to regulate the proliferation and migration of myofibroblasts. FIG. 6A shows the proliferation ability of myofibroblasts transfected with miR-630 mimic detected by RTCA system, and FIG. 6B shows the proliferation ability of myofibroblasts transfected with miR-630 inhibitor detected by RTCA system, FIG. 6C shows the migration ability of myofibroblasts transfected with miR-630 mimic detected by RTCA system, and FIG. 6D shows the migration ability of myofibroblasts transfected with miR-630 inhibitor detected by RTCA system, indicating that the migration ability of myofibroblasts was closely related to the expression of circRNA-0007535.

FIG. 7 shows the technical route provided by the present disclosure.

### DETAILED DESCRIPTION OF THE EMBODIMENTS

The present disclosure provides a use of a reagent for down-regulating the circular RNA-0007535 expression in preparation of drugs for preventing and/or treating pulmonary fibrosis, the nucleotide sequence of the circular RNA-0007535 is set forth in SEQ ID NO. 1. The circular RNA-0007535 gene sequence of the present disclosure is as follow:

TTTCAGAAAGTGCTTTCTCTCTGTGGACATGAGGAT
TGGATTAGAGGAGTGGAATGGGCAGCCTTTGGTAG
AGATCTTTTCCTAGCAAGCTGTTCACAAGATTGCC
TGATAAGAATATGGAAGCTGTATATAAAGTCAACA
TCTTTAGAAACTCAGGATGACGATAACATAAGACT
GAAAGAAAATACTTTTACCATAGAAAATGAAAGTG
TTAAAATAGCATTTGCTGTTACTCTGGAGACAGTG
CTAGCCGGTCATGAAAACTGGGTAAATGCAGTTCA
CTGGCAACCTGTGTTTTACAAAGATGGTGTCCTAC
AGCAGCCAGTGAGATTATTATCTGCTTCCATGGAT
AAAACCATGATTCTCTGGGCTCCAGATGAAGAGTC
AGGAGTTTGGCTAGAACAGGTTCGAGTAGGTGAAG
TAGGTGGGAATACTTTGGGATTTTATGATTGCCAG
TTCAATGAAGATGGCTCCATGATCATTGCTCATGC
TTTCCACGGAGCGTTGCACCTTTGGAAACAGAATA
CAGTTAACCCAAGAGAGTGGACTCCAGAGATTGTC
ATTTCAGGACACTTTGATGGTGTCCAAGACCTAGT
CTGGGATCCAGAAGGAGAATTTATTATCACTGTTG
GTACTGATCAGACAACTAGACTTTTTGCTCCATGG
AAGAGAAAAGACCAATCACAGGTGACTTGGCATGA
AATTGCAAGGCCTCAGATACATGGGTATGACCTGA
AATGTTTGGCAATGATTAATCGGTTTCAGTTTGTA
TCTGGAGCAGATGAAAAAGTTCTTCGGGTTTTTTC
TGCACCTCGGAATTTTGTGGAAAATTTTTGTGCCA
TTACAGGACAATCACTGAATCATGTGCTCTGTAAT
CAAGATAGTGATCTTCCAGAAGGAGCCACTGTCCC
TGCATTGGGATTATCAAATAAAGCTGTCTTTCAGG
GAGATATAGCTTCTCAGCCTTCTGATGAAGAGGAG
CTGTTAACTAGTACTGGTTTTGAGTATCAGCAGGT
GGCCTTTCAGCCCTCCATACTTACTGAGCCTCCCA
CTGAGGATCATCTTCTGCAGAATACTTTGTGGCCT
GAAGTTCAAAAACT.

The present disclosure also provides a use of a reagent for down-regulating the circular RNA-0007535 expression in preparation of drugs for preventing and/or treating pulmonary fibrosis by regulating a Hippo signaling pathway by adsorbing miR-630, and the nucleotide sequence of the circular RNA-0007535 is set forth in SEQ ID NO. 1.

The present disclosure also provides a use of a reagent for down-regulating the circular RNA-0007535 expression in preparation of drugs for inhibiting the differentiation of lung fibroblasts, and the nucleotide sequence of the circular RNA-0007535 is set forth in SEQ ID NO. 1.

The present disclosure also provides a use of a reagent for down-regulating the circular RNA-0007535 expression in preparation of drugs for inhibiting the activation of lung fibroblasts, the nucleotide sequence of the circular RNA-0007535 is set forth in SEQ ID NO. 1.

The present disclosure also provides a use of a reagent for down-regulating the circular RNA-0007535 expression in preparation of drugs for inhibiting the proliferation of lung fibroblasts, and the nucleotide sequence of the circular RNA-0007535 is set forth in SEQ ID NO. 1.

The present disclosure also provides a use of a reagent for down-regulating the circular RNA-0007535 expression in preparation of drugs for inhibiting the migration of lung fibroblasts, and the nucleotide sequence of the circular RNA-0007535 is set forth in SEQ ID NO. 1.

The present disclosure also provides a drug for down-regulating the circular RNA-0007535 expression, wherein the nucleotide sequence of the circular RNA-0007535 is set forth in SEQ ID NO. 1, and the interfering double-stranded RNA is synthesized according to the nucleotide sequence set forth in SEQ ID NO. 2, the drug includes interfering double-stranded RNA, which has the following structure:

```
          5′ GAAGUUCAAAAACUUUUCA dTdT 3′
                |||||||||||||||||||
3′ dTdT CUUCAAGUUUUUGAAAAGU 5′;
```

wherein each “|” represents a base pair.

The above-mentioned interfering double-stranded RNA (interfering sequence) according to the circRNA-0007535 gene is obtained in the present disclosure, specifically, is synthesized according to a target gene whose nucleotide sequence is set forth in SEQ ID NO.2: 5′-GAAGTTCAAAAACTTTTCA-3′, the interfering double-stranded RNA has 19 base pairs, and the first strand and the second strand are respectively designed with two protruding bases dT at the 3′ end.

In the present disclosure, the interference sequence of circRNA-0007535 is specifically targeted through the above design, and transfected into cells to play a role of down-regulating the expression of circRNA-0007535, thereby achieving the purpose of preventing and treating pulmonary fibrosis.

FIG. 7 shows the technical route provided by the present disclosure. In the present disclosure, firstly, the characteristics of circRNA-0007535 are identified, and the clinical value of circRNA-0007535 is clarified. The expression of circRNA-0007535 are knocked down and increased by using the siRNA and over-expression vector respectively, the changes of fibroblasts activation and relevant functional indicators are detected under the intervention of circRNA-0007535. The intracellular localization of circRNA-0007535 is detected by in situ hybridization to determine the regulatory mode of the cell. The binding of circRNA-0007535 to miR-630 is clarified, and the binding relationship is further confirmed by RNA pull down, Ago RIP, and dual luciferase reporter gene assay. The effect of miR-630 on the activation and function of myofibroblasts and the regulatory mechanism of circRNA-0007535 on IPF are investigated, so as to achieve the purpose of preventing and treating pulmonary fibrosis.

The use of the reagent for down-regulating the circular RNA-0007535 expression in preparation of drugs for preventing and/or treating pulmonary fibrosis and drugs for down-regulating the circular RNA-0007535 expression described in the present disclosure will be described below with reference to specific examples. The technical schemes of the present disclosure include but are not limited to the following examples.

Materials: Human blood samples were collected from patients with pulmonary fibrosis who meet the diagnostic criteria and selected as the research objects, according to “An Official ATS/ERS/JRS/ALAT Statement Idiopathic pulmonary fibrosis: evidence-based guidelines for diagnosis and management” issued by the American Association of Respiratory Care, and the control groups were selected in parallel according to gender and age. All patients signed an informed consent form. Fibroblasts (MRC-5) were purchased from the ATCC cell bank; MEM medium and fetal bovine serum (FBS) were purchased from Hyclone, USA; TGF-β1 was purchased from Gibco ThermoFisher; RiboFECT™ CP transfection reagent (Guangzhou RiboBio Co., LTD, China); SYBR Green PCR Master Mix (TAKARA, Dalian, China).

### Example 1

Expression of circRNA-0007535

A: Method

1. Cell culture and grouping: MRC-5 cells were cultured in MEM medium containing 10% fetal bovine serum in an incubator at 37° C. and 5% CO₂ saturated humidity. The cells in the logarithmic growth phase were selected and prepared into single cell suspension, the single cell suspension was inoculated on a six-well culture plate. The blank control group and TGF-β1 stimulation group were set up. When the cells grow to 70%-80% confluency, the blank control group was cultured in serum-free medium to synchronize cell growth. TGF-β1 stimulation group was given a TGF-β1 with the final concentration of 5 nM to stimulate for 72 h to complete the transformation of cells into fibroblasts.

2. qRT-PCR verification and analysis of the expression changes of circRNA-0007535

(1) The total RNA of cells was extracted with RNA extraction reagent Trizol, and the absorbance values (A) of RNA solution at 260 nm and 280 nm were measured with NANO drop 2000 spectrophotometer instrument to determine the purity of the samples. The purity and integrity of the RNA were detected with formaldehyde denatured agarose gel electrophoresis.

(2) cDNA was synthesized by RT (reaction solution was prepared on ice), the extracted cellular RNA was quantified by 1 μg and reversely transcribed according to the following procedure: the reaction system was 10 μL in total, which contained 2.0 μL of 5×GDNA Eraser buffer, 1.0 μL of GDNA Eraser and Total RNA and RNase Free dH₂O, 42° C. and 2 min, the samples were immediately placed on ice for incubation once the reaction is complete; the reaction system was 20 μL in total, which contained 4.0 μL of 5×Prime Scrit Buffer, 1.0 μL of Prime Scrit RT Enzyme Mix I, 1.0 μL of RT Primer Mix and 4.0 μL of RNase Free dH₂O, 37° C., 15 min; 85° C., 5 s; the samples were cooled on ice after reacting at 4° C. for 2 min, the obtained cDNA could be directly used for PCR amplification.

(3) PCR reaction system (the reaction mixture was prepared on ice): the final volume of the PCR reaction system was 20 μL, which contained 7.2 μL of double distilled water; 2 μL of cDNA as template; 0.4 μL of forward primer (F:5′ CCTTTCAGCCCTCCATACTTACT3′, SEQ ID NO. 3); 0.4 μL of reverse primer (R:5′ CCATATTCTTATCAGGCAATCTTGT3′, SEQ ID NO. 4); 10 μL of SYBR. The mixture was centrifuged instantaneously and accumulated at the bottom of a Microtube tube for several seconds; 95° C., 30 s; 95° C., 5 s, 60° C., 20 s, 45 cycles in total, the amplified products were stored in a refrigerator at 4° C., GAPDH was used as an internal reference.

B: Analysis of Results

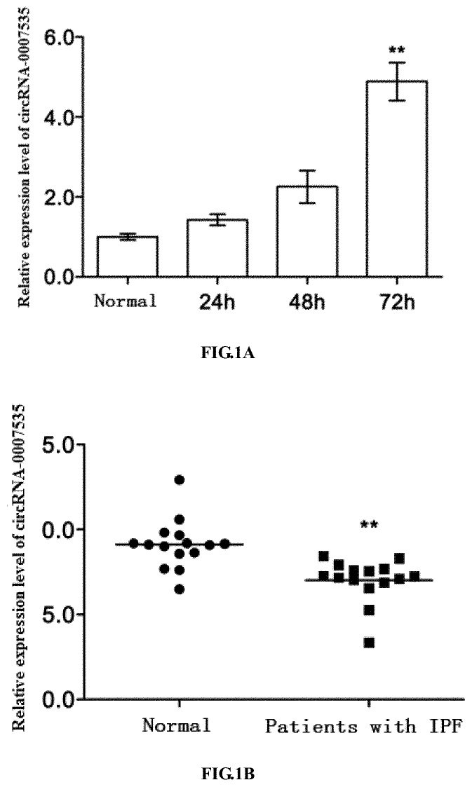

1. The circRNA-0007535 was highly expressed in MRC-5 cells induced and activated by TGF-β1, and the difference was 4.88 times compared with normal cells, indicating that circRNA-0007535 may be involved in regulating the function of fibroblast. The circRNA-0007535 was significantly high expressed in blood samples from patients with IPF, with a difference of 1.30 times compared with normal blood samples (see FIG. 1, in which, FIG. 1A shows that the expression of circRNA-0007535 in MRC-5 cells induced by TGF-β1 is on the rise from 0 h to 72 h through qRT-PCR evaluation, and FIG. 1B verifies the expression level of circRNA-0007535 in blood samples from patients with IPF and normal subjects), indicating that circRNA-0007535 was closely related to the occurrence of pulmonary fibrosis.

### Example 2

Effect of siRNA Interference Fragment Specific for circRNA-0007535 on Transdifferentiation of Fibroblasts

A: Method

1. Cell culture and grouping: MRC-5 cells in logarithmic growth phase were selected and prepared into single cell suspension with 0.25% Trypsin, the single cell suspension was inoculated on a six-well culture plate. When the cells grow to 70%-80% confluency, the siRNA interference fragment, the over-expression plasmid, and the control cells were respectively transfected with riboFECT™ CP transfection reagent in the transfection group and the transfection control group, the final concentration of the siRNA interference fragment and the over-expression plasmid was 50 nM. The blank control group was cultured in serum-free medium, and the model group was stimulated by TGF-β1 with a concentration of 5 nM, the cells were collected after 72 h of culture.

2. The expression changes of pulmonary fibrosis related proteins were detected by Western blot

The protein of each group was extracted in strict accordance with the protocols of Western and IP cell lysate kits, and the protein concentration was determined according to the instructions of BCA protein quantification kit. The protein was separated by 10% SDS-PAGE and transferred to PVDF membrane, the PVDF membrane was taken out and put into the prepared ponceau staining solution to stain for 2 min, the transfer effect was observed. The PVDF membrane was washed twice with TBST for 5 min each time, and was blocked with 7% of (skimmed milk powder+TBST) blocking solution for 2 h at room temperature, the primary antibody was added to incubate overnight at 4° C., and the membrane was washed 3 times with TBST for 5 min each time, the secondary antibodies (1:5000) were added to incubate at room temperature for 60 min; the membrane was washed 3 times with TBST for 15 min each time; the ECL chemiluminescence solution was added for reaction for 3 min, the membrane after reaction was exposed and photographed for observation.

B: Analysis of Results

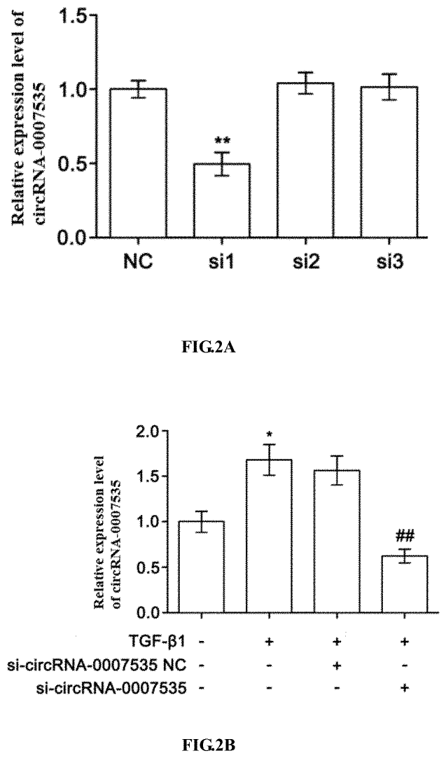

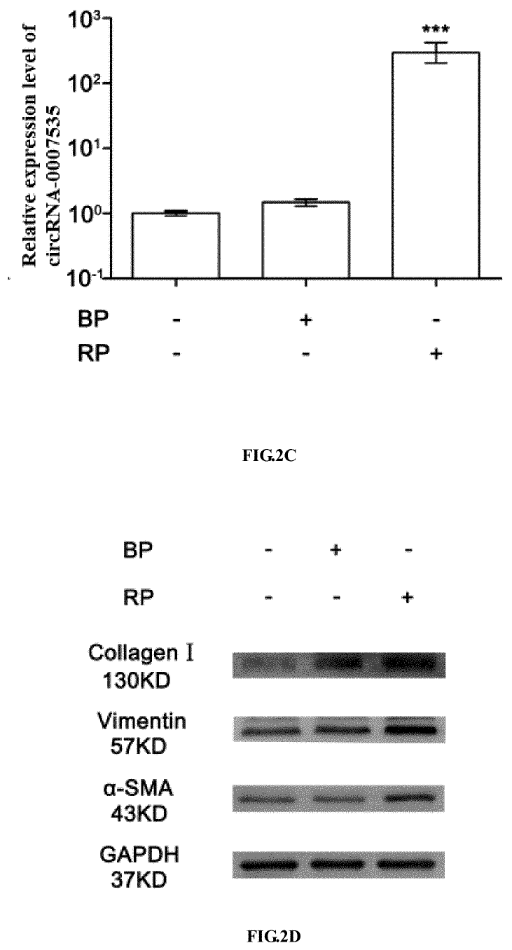

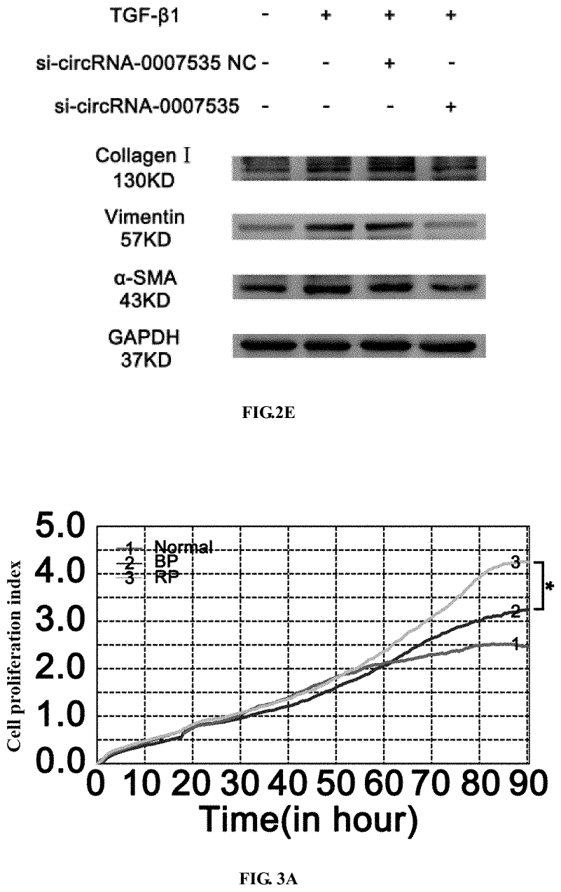

Compared with the blank control group, the expression of pulmonary fibrosis-related proteins in the TGF-β1 stimulation group was significantly increased, indicating that TGF-β1 could induce transformation of cells into myofibroblasts. Compared with the TGF-β1-stimulated group, the expression levels of pulmonary fibrosis-related proteins in the MRC-5/siRNA-0007535 group were significantly down-regulated, indicating that reducing the expression of circRNA-0007535 could inhibit the transdifferentiation of MRC-5 cells (see FIG. 2, in which, FIG. 2A shows the expression level of circRNA-0007535 after different si-circRNA-0007535 transfection, FIG. 2B shows the transfection efficiency of the selected si-circRNA-0007535, FIG. 2C shows the transfection efficiency of the circRNA-0007535 over-expression vector, FIG. 2D shows the changes of pulmonary fibrosis related protein index after the over-expression of circRNA-0007535 detected by western blot, and FIG. 2E shows the changes of pulmonary fibrosis related protein index after the transfection of the interfering fragment si-circRNA-0007535 detected by western blot).

### Example 3

Effects of siRNA Interference Fragment and Over-Expression Plasmid, Specific Targeting circRNA-0007535, on the Proliferation and Migration of Fibroblasts

A: Method

RealTime Cellular Analysis (RTCA) of proliferation and migration: MRC-5 cells in the logarithmic growth phase were selected and prepared into single cell suspension with 0.25% Trypsin, and the single cell suspension was inoculated on six-well culture plate. When the cells grow to 70%-80% confluency, the siRNA interference fragment, the over-expression plasmid, and the control cells were respectively transfected with riboFECT™ CP transfection reagent in the transfection group and the transfection control group, the final concentration of the siRNA interference fragment and the over-expression plasmid was 50 nM. The blank control group was cultured in serum-free medium, and the model group was stimulated by TGF-β1 with a concentration of 5 nM, the cells were collected after 72 h of culture. The collected cells were inoculated on E-Plate and CIM Plate test plates in a detector, and the parameters were set to detect real-time dynamics of cell proliferation and migration within 80 hours, so as to obtain a curve of proliferation and migration.

B: Analysis of Results

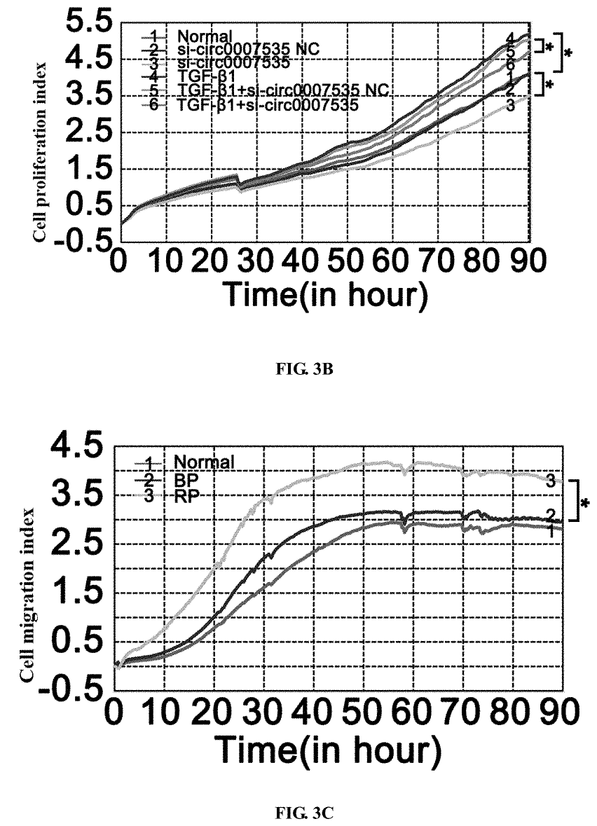

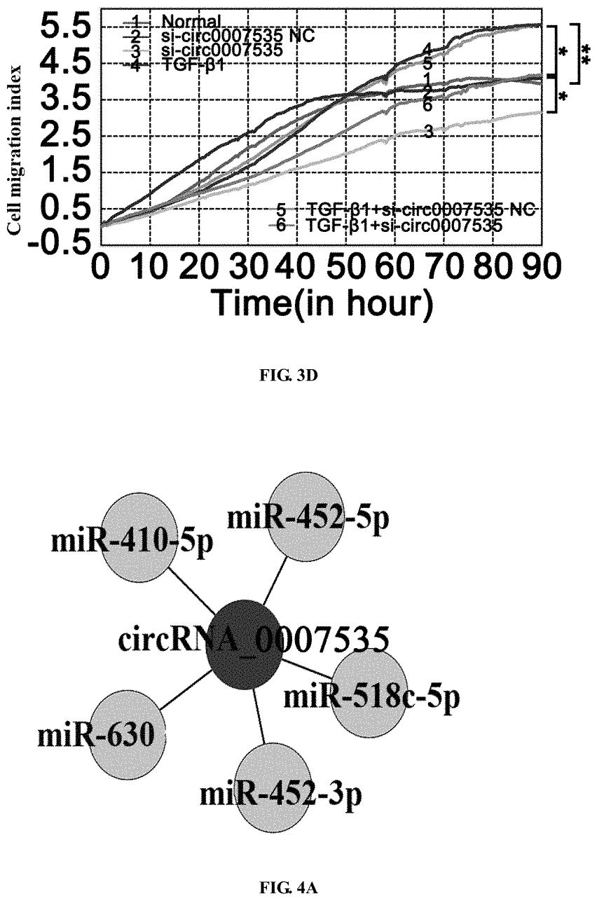

Compared with the TGF-β1 stimulation group, the proliferation and migration abilities of cells in the MRC-5/siRNA-0007535 group were significantly decreased. Compared with the transfected empty plasmid group, the proliferation and migration abilities of the cells transfected with the expression plasmid group were significantly increased (see FIG. 3, in which, FIG. 3A shows the proliferation ability of myofibroblasts after the over-expression of circRNA-0007535 detected by RTCA system, and FIG. 3B shows the proliferation ability of myofibroblasts after the transfection of the interference fragment si-circRNA-0007535 detected by RTCA system, FIG. 3C shows the migration ability of myofibroblasts after over-expression of circRNA-0007535 detected by the RTCA system, and FIG. 3D shows the migration ability of myofibroblasts after transfection of the interfering fragment si-circRNA-0007535 detected by the RTCA system).

### Example 4

The Molecular Mechanism of circRNA-0007535 Regulating the Function of Fibroblasts

A: Method

1. Detection of luciferase reporter gene: the cells were inoculated with a 96-well culture plate upon 70%-80% confluency, and after 24 hours, the reporter gene plasmid and RNA were transfected, and six parawells were set for each sample. The report genes were detected.

2. Cell culture and grouping: MRC-5 cells in logarithmic growth phase were selected and prepared into single cell suspension with 0.25% Trypsin, the single cell suspension was inoculated on a six-well culture plate. When the cells grow to 70%-80% confluency, the miR-630 mimic/inhibitor and the control cells were respectively transfected with riboFECT™ CP transfection reagent in the transfection group and the transfection control group, the final concentration of the siRNA interference fragment and the over-expression plasmid was 50 nM. The blank control group was cultured in serum-free medium, and the model group was stimulated by TGF-β1 with a concentration of 5 nM, the cells were collected after 72 h of culture.

3. qRT-PCR analysis of the expression level of miR-630 in cultured cells: Total RNA was extracted with Trizol reagent, and SYBR Green PCR Master Mix was used for qRT-PCR, and GAPDH were used as an internal reference.

4. The expression changes of pulmonary fibrosis related proteins were detected by Western blot

The protein of each group was extracted in strict accordance with the instructions of Western and IP cell lysate kits, and the protein concentration was determined according to the the instructions of BCA protein quantification kit. The protein was separated by 10% SDS-PAGE and transferred to PVDF membrane, the PVDF membrane was taken out and put into the prepared ponceau staining solution to stain for 2 min, the transfer effect was observed. The PVDF membrane was washed twice with TBST for 5 min each time, and was blocked with 7% of (skimmed milk powder+TBST) blocking solution for 2 h at room temperature, the primary antibody was added to incubate overnight at 4° C., and the membrane was washed 3 times with TBST for 5 min each time, the secondary antibodies (1:5000) were added to incubate at room temperature for 60 min; the membrane was washed 3 times with TBST for 15 min each time; the ECL chemiluminescence solution was added for reaction for 3 min, the membrane after reaction was exposed and photographed for observation.

5. RealTime Cellular Analysis (RTCA) of proliferation and migration: MRC-5 cells in the logarithmic growth phase were selected and prepared into single cell suspension with 0.25% Trypsin, and the single cell suspension was inoculated on six-well culture plate. When the cells grow to 70%-80% confluency, the miR-630 mimic/inhibitor and the control cells were respectively transfected with riboFECT™ CP transfection reagent in the transfection group and the transfection control group, the final concentration of the siRNA interference fragment and the over-expression plasmid was 50 nM. The blank control group was cultured in serum-free medium, and the model group was stimulated by TGF-β1 with a concentration of 5 nM, the cells were collected after 72 h of culture. The collected cells were inoculated on E-Plate and CIM Plate test plates in a detector, and the parameters were set to detect real-time dynamics of cell proliferation and migration within 80 hours, so as to obtain a curve of proliferation and migration.

B: Analysis of Results

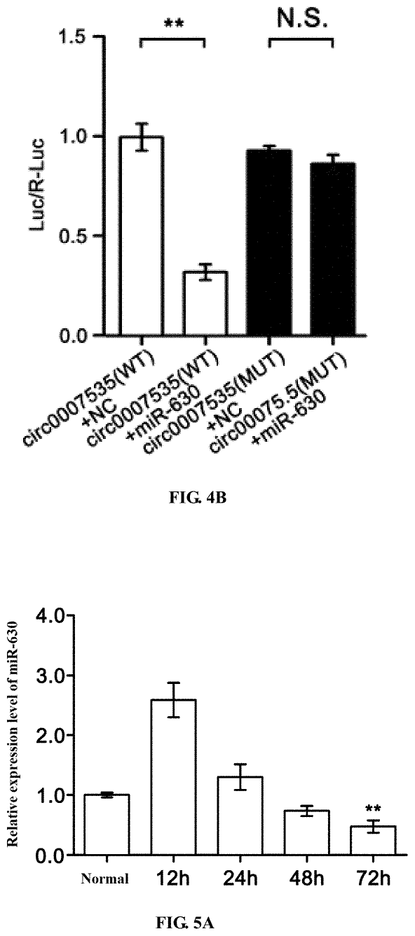

(1) Firefly and Renilla dual Luciferase Reporter Gene Assay shows that miR-630 can inhibit the activity of luciferase combined with circRNA-0007535. After the mutation at the binding site, luciferase activity could not be inhibited by miR-630 (see FIG. 4, in which, FIG. 4A shows the interaction between circRNA-0007535 and its target miRNA in an enlarged network, and FIG. 4B shows that the dual-luciferase report gene detection proved the miR-630 directly binds to circRNA-0007535).

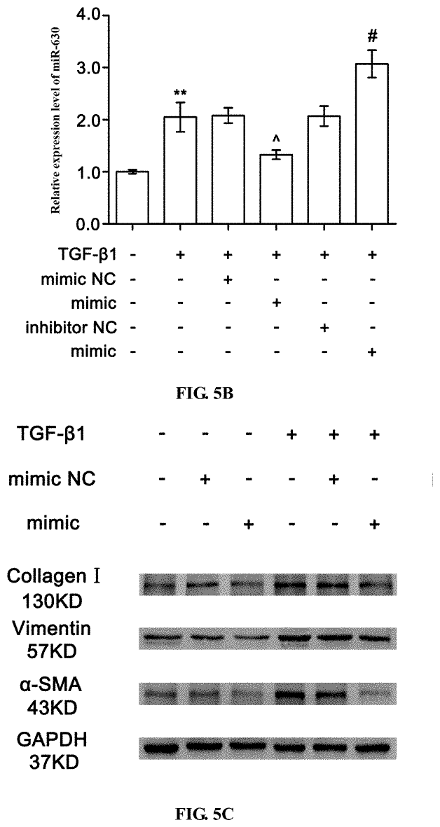

(2) The expression of miR-630 was low in MRC-5 cells activated by TGF-β1; the expression of pulmonary fibrosis related proteins was significantly down-regulated after the addition of miR-630 mimic, the expression level of pulmonary fibrosis related proteins was significantly increased after the addition of miR-630 inhibitor, (see FIG. 5, in which, FIG. 5A shows the expression level of miR-630 in MRC-5 cells induced by TGF-β1 was decreased from 0 h to 72 h evaluated by qRT-PCR, and FIG. 5B shows the expression level of circRNA-0007535 after the addition of miR-630 mimic/inhibitor evaluate by qRT-PCR, FIG. 5C shows the changes of pulmonary fibrosis related protein indicators after transfection of miR-630 mimic detected by Western blot, and FIG. 5D shows the changes of pulmonary fibrosis related protein indicators after transfection of miR-630 inhibitor detected by Western blot), indicating that the expression level of miR-630 was closely related to the expression of circRNA-0007535.

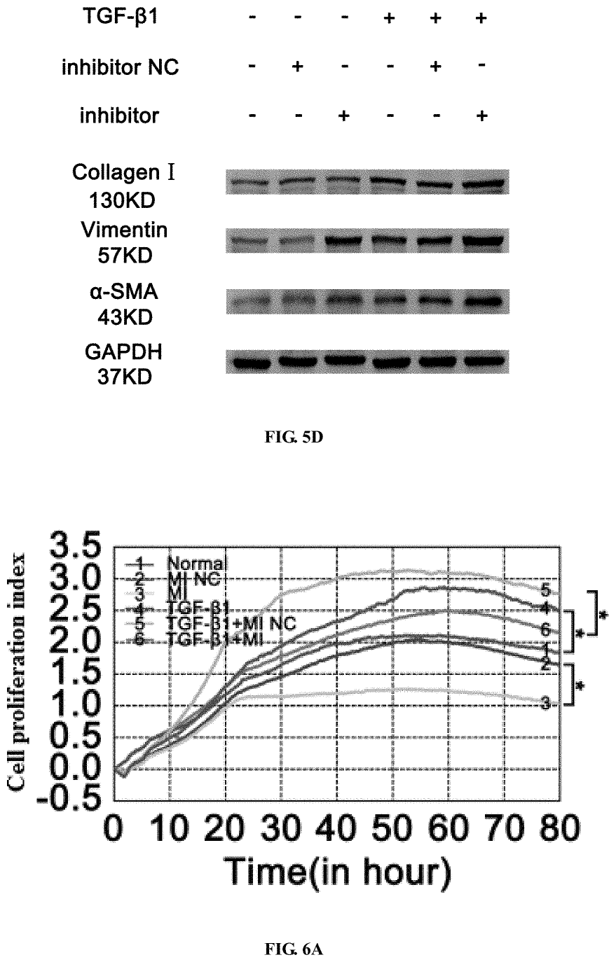

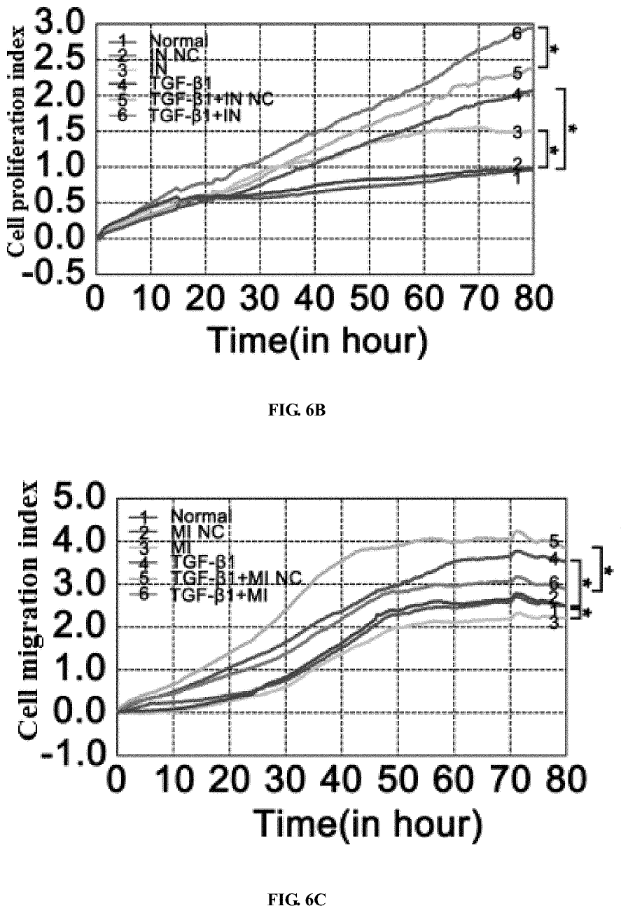

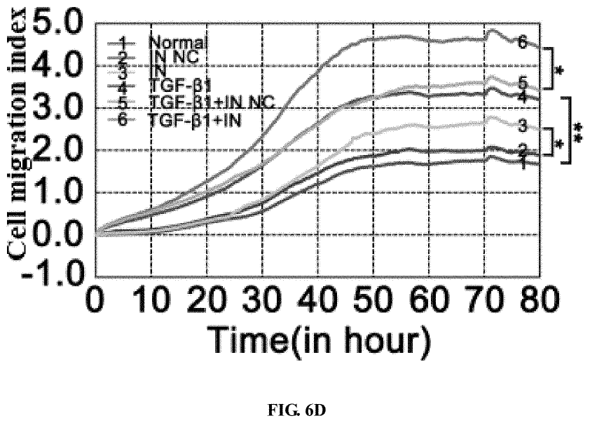

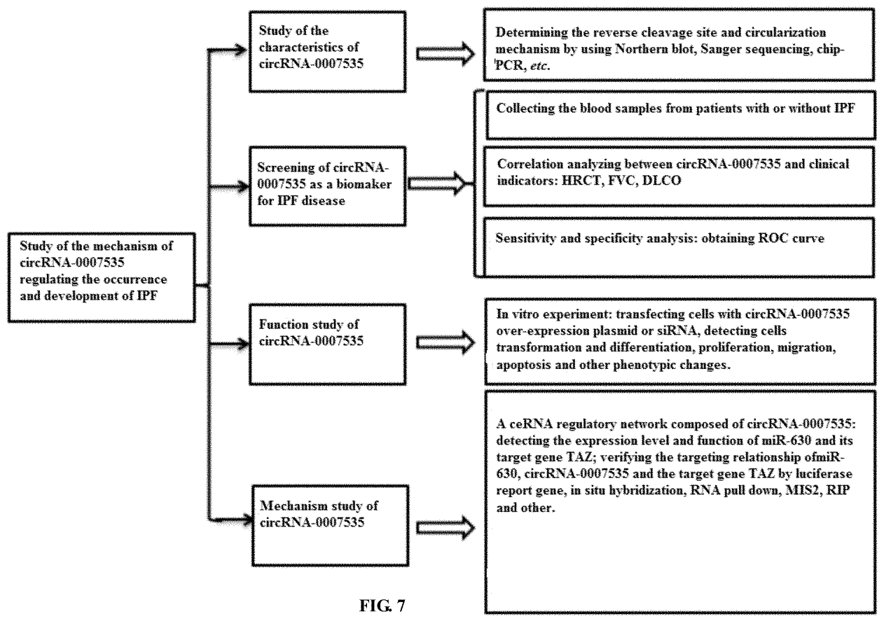

(3) Compared with the TGF-β1 stimulation group, the proliferation and migration abilities of cells in the group after addition of miR-630 mimic were significantly decreased; the proliferation and migration abilities of cells in the group after addition of miR-630 inhibitor was significantly increased (see FIG. 6, in which, FIG. 6A shows the proliferation ability of myofibroblasts transfected with miR-630 mimic detected by RTCA system, and FIG. 6B shows the proliferation ability of myofibroblasts transfected with miR-630 inhibitor detected by RTCA system, FIG. 6C shows the migration ability of myofibroblasts transfected with miR-630 mimic detected by RTCA system, and FIG. 6D shows the migration ability of myofibroblasts transfected with miR-630 inhibitor detected by RTCA system, indicating that the migration ability of myofibroblasts was closely related to the expression of circRNA-0007535.

The above results indicated that reducing the expression of circRNA-0007535 could inhibit the activation, proliferation and migration of fibroblasts, and the circRNA-0007535 may be used as a molecular and drug target for the treatment of pulmonary fibrosis. According to the present disclosure, the interference sequence specific for circRNA-0007535 is designed and transfected into cells to play a role of down-regulating the expression of circRNA-0007535, thereby achieving the purpose of preventing and treating pulmonary fibrosis.

The above described are only preferred embodiments of the present disclosure, it should be noted that, for those skilled in the art, several improvements and retouches can be made without departing from the principles of the present disclosure, and these improvements and retouches also should be regarded as the protection scope of the present disclosure.

1. A method for preventing and/or treating pulmonary fibrosis, comprising administrating a reagent for down-regulating the circular RNA-0007535 expression to a subject in need thereof, wherein the nucleotide sequence of the circular RNA-0007535 is set forth in SEQ ID NO. 1.

2. The method according to claim 1, wherein the reagent prevents and/or treats pulmonary fibrosis by regulating a Hippo signaling pathway by adsorbing miR-630, and the nucleotide sequence of the circular RNA-0007535 is set forth in SEQ ID NO. 1.

3. The method according to claim 1, wherein the reagent inhibits the differentiation of lung fibroblasts, and the nucleotide sequence of the circular RNA-0007535 is set forth in SEQ ID NO. 1.

4. The method according to claim 1, wherein the reagent for inhibits the activation of lung fibroblasts, the nucleotide sequence of the circular RNA-0007535 is set forth in SEQ ID NO. 1.

5. The method according to claim 1, wherein the reagent inhibits the proliferation of lung fibroblasts, and the nucleotide sequence of the circular RNA-0007535 is set forth in SEQ ID NO. 1.

6. The method according to claim 1, wherein the reagent inhibits the migration of lung fibroblasts, and the nucleotide sequence of the circular RNA-0007535 is set forth in SEQ ID NO. 1.

7. The method according to claim 1, wherein the agent for down-regulating the circular RNA-0007535 expression is an interfering double-stranded RNA, the interfering double-stranded RNA is synthesized according to the nucleotide sequence set forth in SEQ ID NO.2, the interfering double-stranded RNA has the following structure:
```
          5′ GAAGUUCAAAAACUUUUCA dTdT 3′
                |||||||||||||||||||
3′ dTdT CUUCAAGUUUUUGAAAAGU 5′;
```
wherein each “|” represents a base pair.

8. A reagent for down-regulating the circular RNA-0007535 expression, wherein the nucleotide sequence of the circular RNA-0007535 is set forth in SEQ ID NO. 1, the reagent is an interfering double-stranded RNA, the interfering double-stranded RNA is synthesized according to the nucleotide sequence set forth in SEQ ID NO.2, the interfering double-stranded RNA has the following structure:
```
          5′ GAAGUUCAAAAACUUUUCA dTdT 3′
                |||||||||||||||||||
3′ dTdT CUUCAAGUUUUUGAAAAGU 5′;
```
wherein each “|” represents a base pair.

9. A drug for down-regulating the circular RNA-0007535 expression, wherein the nucleotide sequence of the circular RNA-0007535 is set forth in SEQ ID NO. 1, the drug comprises an interfering double-stranded RNA, the interfering double-stranded RNA is synthesized according to the nucleotide sequence set forth in SEQ ID NO.2, the interfering double-stranded RNA has the following structure:
```
          5′ GAAGUUCAAAAACUUUUCA dTdT 3′
                |||||||||||||||||||
3′ dTdT CUUCAAGUUUUUGAAAAGU 5′;
```
wherein each “|” represents a base pair.

10. The method according to claim 2, wherein the agent for down-regulating the circular RNA-0007535 expression is an interfering double-stranded RNA, the interfering double-stranded RNA is synthesized according to the nucleotide sequence set forth in SEQ ID NO.2, the interfering double-stranded RNA has the following structure:
```
          5′ GAAGUUCAAAAACUUUUCA dTdT 3′
                |||||||||||||||||||
3′ dTdT CUUCAAGUUUUUGAAAAGU 5′;
```
wherein each “|” represents a base pair.

11. The method according to claim 3, wherein the agent for down-regulating the circular RNA-0007535 expression is an interfering double-stranded RNA, the interfering double-stranded RNA is synthesized according to the nucleotide sequence set forth in SEQ ID NO.2, the interfering double-stranded RNA has the following structure:
```
          5′ GAAGUUCAAAAACUUUUCA dTdT 3′
                |||||||||||||||||||
3′ dTdT CUUCAAGUUUUUGAAAAGU 5′;
```
wherein each “|” represents a base pair.

12. The method according to claim 4, wherein the agent for down-regulating the circular RNA-0007535 expression is an interfering double-stranded RNA, the interfering double-stranded RNA is synthesized according to the nucleotide sequence set forth in SEQ ID NO.2, the interfering double-stranded RNA has the following structure:
```
          5′ GAAGUUCAAAAACUUUUCA dTdT 3′
                |||||||||||||||||||
3′ dTdT CUUCAAGUUUUUGAAAAGU 5′;
```
wherein each “|” represents a base pair.

13. The method according to claim 5, wherein the agent for down-regulating the circular RNA-0007535 expression is an interfering double-stranded RNA, the interfering double-stranded RNA is synthesized according to the nucleotide sequence set forth in SEQ ID NO.2, the interfering double-stranded RNA has the following structure:
```
          5′ GAAGUUCAAAAACUUUUCA dTdT 3′
                |||||||||||||||||||
3′ dTdT CUUCAAGUUUUUGAAAAGU 5′;
```
wherein each “|” represents a base pair.

14. The method according to claim 6, wherein the agent for down-regulating the circular RNA-0007535 expression is an interfering double-stranded RNA, the interfering double-stranded RNA is synthesized according to the nucleotide sequence set forth in SEQ ID NO.2, the interfering double-stranded RNA has the following structure:
```
          5′ GAAGUUCAAAAACUUUUCA dTdT 3′
                |||||||||||||||||||
3′ dTdT CUUCAAGUUUUUGAAAAGU 5′;
```
wherein each “|” represents a base pair.
